(19) United States
(12) Patent Application Publication
(10) Pub. No.: US 2023/0131248 A1
(43) Pub. Date: Apr. 27, 2023

(54) A SKIN TEST PLATFORM FORMED FROM A COMPOSITE MATERIAL

(71) Applicant: Nanyang Technological University, Singapore (SG)

(72) Inventors: Yajing CUI, singapore (SG); Geng CHEN, Singapore (SG); Xiangdong CHEN, Singapore (SG); Suxuan GONG, Singapore (SG)

(21) Appl. No.: 17/801,678

(22) PCT Filed: Mar. 11, 2021
(86) PCT No.: PCT/SG2021/050128

§ 371 Date: Aug. 23, 2022

(30) Foreign Application Priority Data
Mar. 11, 2020 (SG) 10202002240P

(51) Int. Cl. A61L 27/60; A61L 27/22; A61L 27/56

(52) U.S. Cl. CPC A61L 27/60; A61L 27/227; A61L 27/56

(57) ABSTRACT

Disclosed herein is a composite material and a skin test platform material in a form of a membrane, comprising silk fibroin and a crosslinking agent, wherein from 4.7 to 14 wt % of the total dry weight of the material is derived from the crosslinking agent. In one embodiment, the crosslinking agent is polyethylene glycol) diglycidyl ether. The membrane has a surface that may be shaped to mimic human skin structures. Also disclosed herein are methods of forming a composite material, a skin test platform material, and determining a property of a test composition such as an anti-bacterial cleansing composition, a skin care product and a perfume.

### FIELD OF INVENTION

The current invention relates to a skin test platform material and the underlying composite material per se. The invention also relates to methods of making and using said materials.

### BACKGROUND

The listing or discussion of a prior-published document in this specification should not necessarily be taken as an acknowledgement that the document is part of the state of the art or is common general knowledge.

A key concern in the cosmetic industry is the absorption of skincare products into the lipid and protein layers of skin. Human skin is the best platform to test this factor but is rarely available due to ethical and other considerations. Generally, the R&D department in cosmetics companies employ a routine skin model test to obtain the products' absorption rates. In this absorption test, researchers apply skincare products to the skin model, and evaluate the condition of the model after, to determine whether the products resulted in certain improvement effect such as moisturizing, self-tanning, whitening, etc. to the skin model.

By far, there is limited choices of materials that are suitable for building skin model to mimic skin properties such as skin humidity conditions and absorbability. Current skin models either use metals which exhibit low absorbability to water/emulsion, or other available materials such as vitroskin and transpore tape that are too expensive. Traditional methods using PMMA substrate to make skin model is far from scientific as the surface properties of PMMA is significantly different from real skin. In addition, as a large amount of this kind of substrate is needed to make the skin model, the commercially available human skin-like product is too expensive which increases the expenditure of the cosmetic companies. As a result, the demand for high-quality and human skin-like substrate is currently meeting a dilemma in most cosmetic companies.

Therefore, there is a need to develop a new and suitable in vitro skin model that mimics the skin absorption behaviour by directly absorbing small molecules (for e.g., molecules whose molecular weight are lower than or about 500 Da) but excludes bulky molecules (for e.g. molecules whose molecular weight are higher than 500 Da). In addition, this new substitute must have a competitive cost as compared to the currently available products.

### SUMMARY OF INVENTION

Aspects and embodiments of the invention will now be described by reference to the following numbered clauses.

1 A skin test platform material in the form of a membrane, said material comprising:
silk fibroin; and
a crosslinking agent that forms crosslinks in the silk fibroin, wherein
from 4.7 to 14 wt % of the total dry weight of the skin test platform material is derived from the crosslinking agent.

2. The skin test platform material according to Clause 1, wherein the membrane has a surface that has been shaped to mimic human skin structures.

3. The skin test platform material according to Clause 1 or Clause 2, wherein the membrane has a plurality of pores, optionally wherein:
each pore independently has a diameter of from 95 to 500 μm, such as 99 to 250 μm; and/or
the plurality of pores has a pore density of from 50 to 80 pores cm⁻².

4. The skin test platform material according to any one of the preceding clauses, wherein the crosslinking agent is selected from one or more of the group consisting of O′O-bis[2-(N-succinimidyl succinylamino) ethyl]polyethylene glycol (NHSP), glutaraldehyde, poly(ethylene glycol) dithiol, a 4-arm PEG-thiol, a 4-arm PEG-epoxide, a 4-arm PEG isocyanate, isocyanate PEG isocyanate, poly(ethylene glycol) diglycidyl ether (e.g. poly(ethylene glycol) dithiol, a 4-arm PEG-thiol, a 4-arm PEG-epoxide, a 4-arm PEG isocyanate, isocyanate PEG isocyanate, and poly(ethylene glycol) diglycidyl ether.

5. The skin test platform material according to Clause 4, wherein the crosslinking agent is poly(ethylene glycol) diglycidyl ether.

6. The skin test platform material according to Clause 4 or Clause 5, wherein when the crosslinking agent is a polymeric material it has a number average molecular weight of from 150 to 2,000 Daltons, such as from 150 to 1,000 Daltons, such as from 200 to 700 Daltons, such as from 250 to 500 Daltons.

7. The skin test platform material according to any one of the preceding clauses, wherein:

(az) the membrane is from 50 to 500 μm, such as from 60 to 450 μm, such as from 70 to 400 μm, such as about 74 μm, such as about 75 μm, such as about 400 μm thick; and/or

(bz) from 6.5 to 11 wt %, such as from 9 to 10 wt %, of the total dry weight of the skin test platform material is derived from the crosslinking agent.

8. The skin test platform material according to any one of the preceding clauses, wherein the skin test platform further comprises an additive material, optionally wherein the additive material is poly(ethylene glycol) diacrylate.

9. The skin test platform material according to any one of the preceding clauses, wherein one or more of the following apply:

(a) the silk fibroin is derived from Bombyx mori silkworm silk;

(b) the skin test platform material can be subjected to an elongation strain of from 300 to 550% without breaking, such as from 350 to 500% without breaking;

(c) the skin test platform material has a Young's modulus of from 2.5 to 10 MPa, such as from 5 to 8 MPa, such as about 6.5 MPa;

(d) when the skin test platform material has not been shaped to mimic human skin structures, it has a water contact angle at 30 seconds after contact with water of from 60 to 70°;

(e) when the skin test platform material has been shaped to mimic human skin structures, it has a water contact angle at 30 seconds after contact with water of from 30 to 40°;

(f) a difference between a water contact angle of the skin test platform material that has not been shaped to mimic human skin structures and a water contact angle of the same skin test platform material that has been shaped to mimic human skin structures at 30 seconds after contact with water is from 20° to 40°, such as about 30°; and

(g) when the skin test platform material has not been shaped to mimic human skin structures, it has a water contact angle upon immediate contact with water of from 80 to 90°.

10. A composite material comprising:
silk fibroin; and
a crosslinking agent that forms crosslinks in the silk fibroin, wherein
from 4.7 to 14 wt % of the total dry weight of the composite material is derived from the crosslinking agent.

11. The composite material according to Clause 10, wherein the composite material is formed as a membrane, optionally wherein the membrane has one or both of the following features:

a surface that has been shaped to mimic human skin structures; and

a surface with a plurality of pores, further optionally wherein when the surface has a plurality of pores:

each pore independently has a diameter of from 95 to 500 μm, such as 99 to 250 μm; and/or

the plurality of pores has a pore density of from 50 to 80 pores cm⁻².

12. The composite material according to Clause 10 or Clause 11, wherein the crosslinking agent is selected from one or more of the group consisting O′O-bis[2-(N-succinimidyl succinylamino) ethyl]polyethylene glycol (NHSP), glutaraldehyde, poly(ethylene glycol) dithiol, a 4-arm PEG-thiol, a 4-arm PEG-epoxide, a 4-arm PEG isocyanate, isocyanate PEG isocyanate, and poly(ethylene glycol) diglycidyl ether (e.g. poly(ethylene glycol) dithiol, a 4-arm PEG-thiol, a 4-arm PEG-epoxide, a 4-arm PEG isocyanate, isocyanate PEG isocyanate, and poly(ethylene glycol) diglycidyl ether).

13. The composite material according to Clause 12, wherein the crosslinking agent is poly(ethylene glycol) diglycidyl ether.

14. The composite material according to Clause 12 or Clause 13, wherein:

(aaz) when the crosslinking agent is a polymeric material it has a number average molecular weight of from 150 to 2,000 Daltons, such as from 150 to 1,000 Daltons, such as from 200 to 700 Daltons, such as from 250 to 500 Daltons; and/or

(bbz) from 6.5 to 11 wt %, such as from 9 to 10 wt %, of the total dry weight of the composite material is derived from the crosslinking agent.

15. The composite material according to any one of Clauses 10 to 14, wherein the composite material further comprises an additive material, optionally wherein the additive material is poly(ethylene glycol) diacrylate.

16. The composite material according to any one of Clauses 10 to 15, wherein one or more of the following apply:

(ai) the silk fibroin is derived from Bombyx mori silkworm silk;

(bi) when the composite material is provided as a membrane, the composite material can be subjected to an elongation strain of from 300 to 550% without breaking, such as from 350 to 500% without breaking;

(ci) when the composite material is provided as a membrane, the composite material has a Young's modulus of from 2.5 to 10 MPa, such as from 5 to 8 MPa, such as about 6.5 MPa;

(di) when the composite material is provided as a membrane, but has not been shaped to mimic human skin structures, it has a water contact angle at 30 seconds after contact with water of from 60 to 70°;

(ei) when the composite material is provided as a membrane and has been shaped to mimic human skin structures, it has a water contact angle at 30 seconds after contact with water of from 30 to 40°;

(fi) a difference between a water contact angle of the composite material provided as a membrane that has not been shaped to mimic human skin structures and a water contact angle of the same composite material provided as a membrane that has been shaped to mimic human skin structures at 30 seconds after contact with water is from 20° to 40°, such as about 30°;

(gi) the composite material in the form of a membrane has a thickness of from 50 to 500 μm, such as from 60 to 450 μm, such as from 70 to 400 μm, such as about 74 μm, such as about 75 μm, such as about 400 μm; and

(hi) when the composite material is provided as a membrane and not been shaped to mimic human skin structures, it has a water contact angle upon immediate contact with water of from 80 to 90°.

17. A method of forming a composite material according to any one of Clauses 10 to 16, comprising the steps of:

(ia) providing a solution comprising an extracted silk fibroin and a solvent; and

(ib) adding from 5 to 15 wt %, based upon the dry weight of the extracted silk fibroin, of a crosslinking agent to the silk fibroin solution to form the desired composite material by reaction with the silk fibroin.

18. A method of forming a skin test platform material in the form of a membrane according to any one of Clauses 1 to 9, comprising the steps of:

(A) providing a solution comprising an extracted silk fibroin and a solvent;

(B) adding from 5 to 15 wt %, based upon the dry weight of the extracted silk fibroin, of a crosslinking agent to the silk fibroin solution to form the skin test platform material in solution by reaction with the silk fibroin;

(C) casting the skin test platform material in solution into a mould and evaporating the solvent to provide the skin test platform material in the form of a membrane.

19. The method according to Clause 18, wherein the mould has a surface that has been shaped to mirror human skin structures, so as to produce a membrane that has a surface that mimics human skin structures.

20. The method according to Clause 17 or the method according to Clauses 18 or 19, wherein the method further comprises applying a plurality of pores to the membrane, optionally wherein:

each pore independently has a diameter of from 95 to 500 μm, such as 99 to 250 μm; and/or

the plurality of pores has a pore density of from 50 to 80 pores cm⁻².

21. The method according to Clause 17 and Clause 20 as dependent upon Clause 17 or the method according to any one of Clause 18 and Clause 19 to 20 as dependent on Clause 18, wherein the crosslinking agent is selected from one or more of the group consisting of O′O-bis[2-(N-succinimidyl succinylamino) ethyl]polyethylene glycol (NHSP), glutaraldehyde, poly(ethylene glycol) dithiol, a 4-arm PEG-thiol, a 4-arm PEG-epoxide, a 4-arm PEG isocyanate, isocyanate PEG isocyanate, and poly(ethylene glycol) diglycidyl ether (e.g. poly(ethylene glycol) dithiol, a 4-arm PEG-thiol, a 4-arm PEG-epoxide, a 4-arm PEG isocyanate, isocyanate PEG isocyanate, or more particularly, poly(ethylene glycol) diglycidyl ether), optionally wherein one or more of the following apply:

(Ai) the crosslinking agent is poly(ethylene glycol) diglycidyl ether;

(Aii) when the crosslinking agent is a polymeric material it has a number average molecular weight of from 150 to 2,000 Daltons, such as from 150 to 1,000 Daltons, such as from 200 to 700 Daltons, such as from 250 to 500 Daltons;

(Aiii) the crosslinking agent is added to the solution comprising an extracted silk fibroin and a solvent in an amount of from 7 to 12 wt %, such as about 10 wt %, relative to the dry weight of the extracted silk fibroin.

22. The method according to any one of Clauses 17 and 20 to 21 as dependent upon Clause 17 or the method according to any one of Clauses 18 and 19 to 21 as dependent on Clause 18, wherein the reaction of the crosslinking agent with the silk fibroin is effected using ultrasonication and/or heating for a period of time, optionally wherein:
when used, the heating is from 40 to 80° C., such as from 50 to 70° C., such as about 60° C.; and/or
the period of time is from 10 minutes to 24 hours, such as from 20 minutes to 12 hours, such as from 25 minutes to 1 hour, such as about 30 minutes.

23. The method according to any one of Clauses 17 and 20 to 22 as dependent upon claim 17 or the method according to any one of Clauses 18 and 19 to 22 as dependent on claim 18, wherein the method further comprises adding an additive material to the composite material formed in step (ib) of Clause 17 or the skin test platform material in solution formed in step (B) of Clause 18, optionally wherein the additive material is poly(ethylene glycol) diacrylate.

24. A method of determining a property of a test composition, the method comprising the steps of:

(zi) supplying a skin test platform as described in any one of Clauses 1 to 9;

(zii) applying a test composition to the skin test platform and determining a property of said composition.

25. The method according to Clause 24, wherein the composition and property is selected from one of:

(zzi) the composition is an anti-bacterial cleansing composition and step (zii) of Clause 24 comprises applying the composition to the skin test platform, subsequently rinsing it off and then applying a bacterial culture to the skin test platform and measuring the growth of the bacterial culture on the skin test platform to determine an antibacterial activity of the anti-bacterial cleansing composition;

(zzii) the composition is a skincare product and step (zii) of Clause 24 comprises applying the composition to a portion of the skin test platform and then measuring a dynamic friction force of the composition on the portion where the skincare composition was applied; or

(zziii) the composition is a perfume and step (zii) of Clause 24 comprises applying the composition to a portion of the skin test platform and then measuring a release profile of the perfume over time.

1) Tests for anti-bacteria performance of hand wash/hand sanitizer/shower gel by applying the skin products and culture bacteria on the skin test platform.

2) Tests for absorbency and sensory feel after products application by applying the skincare products and measure the dynamic friction force of the applied area.

3) Tests for the release profile of perfume applied on the skin test platform.

### DRAWINGS

Aspects and embodiments of the invention are illustrated by the following drawings.

FIG. 1 depicts the FTIR results of reacted PEGDE-SF and simply-mixed poly(ethylene glycol) diglycidyl ether (PEGDE) and silk fibroin (SF).

FIG. 2 shows a) FTIR spectra of silk treated with different weight ratios of PEGDE show ether moiety (1055 cm⁻¹) is present in silk and the secondary structure of silk shift from the crystalline domain (1511 cm⁻¹) to the amorphous domain (1535 cm⁻¹) and back to the crystalline domain (1511 cm⁻¹) with increasing PEGDE weight ratio; and b)¹H NMR of PEGlyated SF (PSF) and pristine SF showing the engraftment of ethylene glycol moiety to silk fibroin.

FIG. 3 shows a) optical image of PEGDE/SF substrate with stratum corneum structure; b) contact angle test of PEGDE/SF substrate with and without stratum corneum structure (also referred to as skin structures); and c) measurement of contact angle of PEGDE/SF substrate without stratum corneum structure).

FIG. 4 shows a) mechanical performance comparison of SF-PEGDE reacted via ultrasonication (ultrasonication reacted) and stirring at elevated temperature (heat and stir); and b) control mechanical tests of SF being sonicated with PEGDE (PEGDE-SF), sonicated with PEG-400 (SF-PEG400), and simply-mixed with PEGDE (Simply-mixed SF+PEGDE).

FIG. 5 shows the surface contact angle distribution towards water of PEGDE/SF with stratum corneum structure with different PEGDE ratio.

FIG. 6 depicts the surface contact angle range for varying reacted silk solution concentration.

FIG. 7 depicts the surface contact angle range for PEGDE/SF with stratum corneum structure.

FIG. 8 depicts (a) the results of the mechanical tests of PEGDE-SF with different weight ratio of PEGDE to SF; and (b) the effect of different weight ratio of PEGDE to SF on Young's modulus.

FIG. 9 shows (a-b) possible pore sizes in a photograph of a membrane according to the current invention; (c) a photograph showing a possible pore pattern; and (d) a pore pattern schematically.

FIG. 10 shows the time evolution of water contact angle change of PEGDE/SF with and without pores.

FIG. 11 shows the toner (toner sample provided by P&G SG) absorption process of skin artificial epidermis (i.e. PEGDE-SF without stratum corneum structure) over time: (a) 0 min; (b) 5 min; and (c) 20 min.

FIG. 12 shows the cream (cream sample provided by P&G SG) absorption process of skin artificial epidermis over time: (a) 0 min; (b) 5 min; and (c) 20 min.

FIG. 13 depicts (a) absorbency of lotion by silk artificial epidermis; and (b) the contact angle change of silk artificial epidermis after the application of lotions.

FIG. 14 shows a) the penetration of niacinamide into silk artificial epidermis at different depth; and b) the penetration of glycerin into silk artificial epidermis at different depth.

FIG. 15 depicts the bacterial culture performance of silk artificial epidermis and commercially available artificial epidermis.

FIG. 16 shows the absorption of water on silk artificial epidermis and commercial product VITRO-CORNEUM® in 0 s, 5 s, 10 s, 15 s and 20 s.

FIG. 17 shows (a) a photograph of large-scale silk artificial epidermis; and (b) a photograph of large-scale silk artificial epidermis that shows high transparency.

### DESCRIPTION

Surprisingly, it has been found that a composite material formed from silk fibroin and a crosslinking agent that can crosslink silk fibroin may be used to form a skin test platform that have properties similar to, if not in some cases identical to, skin. Thus, in a first aspect of the invention, there is provided a skin test platform material in the form of a membrane, said material comprising:
silk fibroin; and
a crosslinking agent that forms crosslinks in the silk fibroin, wherein
from 4.7 to 14 wt % of the total dry weight of the skin test platform material is derived from the crosslinking agent.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

When used herein, the term “membrane” will be understood to refer to a material in a form resembling a film. This film/membrane may have any suitable thickness, but may be shaped to have a thickness resembling human skin and/or commercial skin replacement products that are known. For example, the membrane may be from 5 to 4,000 μm, such as from 10 to 1,000 μm, such as from 20 to 100 μm, such as about 30 μm. In particular embodiments that may be mentioned herein, the membrane may be from 50 to 500 μm, such as from 60 to 450 μm, such as from 70 to 400 μm, such as about 74 μm, such as about 75 μm, such as about 400 μm thick.

The skin test platform material may or may not be patterned such that at least one surface of the membrane has been shaped to mimic human skin structures. Examples of how the membrane may be patterned to mimic human skin structures are described in detail below in the examples. However, a liquid form (e.g. a solution) of the material used to form the membrane may be placed into a mould that has been formed from taking an impression of the outer layers of human skin, with the solvent in the solution allowed to evaporate to form the patterned membrane. When used herein, the term “human skin structures” may mean structures found on the surface of the stratum corneum, outermost layer of the epidermis. For example, human skin structures that may be mimicked include dead keratinocytes, pores and the like.

The membrane (with or without being patterned to mimic human skin structures) may also be treated to provide a plurality of pores. These pores may extend part-way or fully through the membrane and may be made by any suitable method. For example, the pores may be made by laser patterning. Each pore may have any suitable diameter and the plurality of pores may have any suitable density. A suitable density for the plurality of pores may mimic the density of pores found in human skin, while the diameter of each pore may also mimic the diameters of pores found in human skin. For example, each pore may independently have a diameter of from 95 to 500 μm, such as 99 to 250 μm. The plurality of pores may have a pore density of from 50 to 80 pores cm⁻². It will be appreciated that the pore diameter and the pore density are not necessarily linked and that there is a great degree of variability in the pore diameters and pore densities that may be used in an embodiment of the invention where pores are present in the membranes discussed herein.

The silk fibroin may be in any suitable hydrogel form. For example, the majority of the silk fibroin may be provided in the form of random coils and turns, though other forms of silk fibroin may also be present (e.g. β-sheets and helixes). For example, in certain embodiments mentioned herein, the silk fibroin may contain about 54.6% random coils and about 45.4% random turns.

In the skin test platform material, the crosslinking agent is provided in a form that maintains a crosslink, either between different silk fibroin protein strands or potentially between different portions of the same strand. The crosslinking agent may be provided in any suitable form. For example, the crosslinking agent could potentially be provided in the form of an ionic material that enables the formation of ionic crosslinks. In particular embodiments that may be mentioned herein, the crosslinking agent may be a material that is able to form covalent bonds with amino acid residues in silk fibroin. For example, the crosslinking agent may be able to form a covalent bond with amino groups or phenol groups in the amino acid residues of silk fibroin. For example, amino acids that may be present in silk fibroin bearing an amino group include Asn, Lys and Pro, while amino acids that may be present in silk fibroin bearing a phenol group include Tyr. One or both of amino groups and phenol groups in the silk fibroin may be used to form the covalent bonds with a crosslinking agent suitable to form covalent bonds.

Any suitable degree of crosslinking of the crosslinkable functional groups may be used herein. For example, the degree of crosslinking of amino (NH₂) groups in the side-chains of amino acids in silk fibroin may be from 1% to 100%, such as from 10% to 99%, such as from 20% to 98%, or more particularly from 33% to 97.7%. The degree of crosslinking of phenol groups in silk fibroin may be from 1% to 100%, such as from 10% to 99%, such as from 15% to 90%, or more particularly from 33% to 88% (e.g. from 15% to 83%). These ranges may apply to any of the aspects and embodiments of the invention described herein.

For the avoidance of doubt, it is explicitly contemplated that where a number of numerical ranges related to the same feature are cited herein, that the end points for each range are intended to be combined in any order to provide further contemplated (and implicitly disclosed) ranges. For example, the degree of crosslinking may be selected from any one of:

from 1 to 10%, such as from 1 to 15%, such as from 1 to 20%, such as from 1 to 33%, such as from 1 to 83%, such as from 1 to 88%, such as from 1 to 90%, such as from 1 to 97.7%, such as from 1 to 99%, such as from 1 to 100%;

such as from 10 to 15%, such as from 10 to 20%, such as from 10 to 33%, such as from 10 to 83%, such as from 10 to 88%, such as from 10 to 90%, such as from 10 to 97.7%, such as from 10 to 99%, such as from 10 to 100%;

from 15 to 20%, such as from 15 to 33%, such as from 15 to 83%, such as from 15 to 88%, such as from 15 to 90%, such as from 15 to 97.7%, such as from 15 to 99%, such as from 15 to 100%;

from 20 to 33%, such as from 20 to 83%, such as from 20 to 88%, such as from 20 to 90%, such as from 20 to 97.7%, such as from 20 to 99%, such as from 20 to 100%;

from 33 to 83%, such as from 33 to 88%, such as from 33 to 90%, such as from 33 to 97.7%, such as from 33 to 99%, such as from 33 to 100%;

from 83 to 88%, such as from 83 to 90%, such as from 83 to 97.7%, such as from 83 to 99%, such as from 83 to 100%;

from 88 to 90%, such as from 88 to 97.7%, such as from 88 to 99%, such as from 88 to 100%;

from 90 to 97.7%, such as from 90 to 99%, such as from 90 to 100%;

from 97.7 to 99%, such as from 97.7 to 100%; and

from 99 to 100%.

Crosslinking agents that may be mentioned herein include, but are not limited to O′O-bis[2-(N-succinimidyl succinylamino) ethyl]polyethylene glycol (NHSP), glutaraldehyde, poly(ethylene glycol) dithiol, a 4-arm PEG-thiol, a 4-arm PEG-epoxide, a 4-arm PEG isocyanate, isocyanate PEG isocyanate, or more particularly poly(ethylene glycol) diglycidyl ether. For example, the crosslinking agent may be poly(ethylene glycol) dithiol, a 4-arm PEG-thiol, a 4-arm PEG-epoxide, a 4-arm PEG isocyanate, isocyanate PEG isocyanate, or more particularly poly(ethylene glycol) diglycidyl ether. For example, the crosslinking agent may be poly(ethylene glycol) diglycidyl ether.

In embodiments of the invention, an additive material may be included in the skin test platform material. For example, the additive material may be poly(ethylene glycol) diacrylate. In embodiments of the invention where poly(ethylene glycol) diacrylate is present as an additive, it may be provided as a surface wettability modifier.

When the crosslinking agent is a polymeric material, it may have any suitable number average molecular weight. For example, when the crosslinking agent is a polymeric material it has a number average molecular weight of from 150 to 2,000 Daltons, such as from 150 to 1,000 Daltons, such as from 200 to 700 Daltons, such as from 250 to 500 Daltons.

As noted above, from 4.7 to 14 wt % of the total dry weight of the skin test platform material is derived from the crosslinking agent. When used herein “total dry weight” refers to the dry weight of the skin test platform material (or to the composite material mentioned herein too). With reference to the method of manufacture, from 5 to 15 wt % of a crosslinking agent relative to the weight of silk fibroin is added to said silk fibroin to form the desired product. This means that the crosslinking agent may form from 4.7 to 14 wt % of the resulting product's dry weight. In embodiments of the invention that may be mentioned herein, from 6.5 to 11 wt %, such as from 9 to 10 wt %, of the total dry weight of the skin test platform material (or other form of the material that may be mentioned herein, such as a composite material) may be derived from the crosslinking agent, which may be interpreted accordingly to the above definitions.

The silk fibroin used in the skin test platform may be obtained from any suitable source. For example, the silk fibroin may be obtained from larvae of Bombyx mori, and other moth genera such as Antheraea, Cricula, Samia and Gonometa. Silk fibroin may also be obtained from spider silk. For example, the silk fibroin may be derived from Bombyx mori silkworm silk.

The skin test platform material may be able to withstand mechanical strain applied to it. For example, the skin test platform material may be subjected to an elongation strain of from 300 to 550% without breaking, such as from 350 to 500% without breaking. The skin test platform material may also, or additionally, display a Young's modulus of from 2.5 to 10 MPa, such as from 5 to 8 MPa, such as about 6.5 MPa.

When the skin test platform material has not been shaped to mimic human skin structures, it may have a water contact angle at 30 seconds after contact with water of from 60 to 70°.

Additionally or alternatively, when the skin test platform material has not been shaped to mimic human skin structures, it may have a water contact angle upon immediate contact with water of from 80 to 90°. It has been surprisingly found that the skin test platform material that has not been shaped to mimic human skin structures may more accurately match the profile of facial skin.

Alternatively, when the skin test platform material has been shaped to mimic human skin structures, it may have a water contact angle at 30 seconds after contact with water of from 30 to 40°.

For example, a difference between a water contact angle of the skin test platform material that has not been shaped to mimic human skin structures and a water contact angle of the same skin test platform material that has been shaped to mimic human skin structures at 30 seconds after contact with water may be from 20° to 40°, such as about 30°.

The skin test platform above makes use of a material formed from crosslinked silk fibroin. As such, in a further aspect of the invention, there is also disclosed a composite material comprising:
silk fibroin; and
a crosslinking agent that forms crosslinks in the silk fibroin, wherein
from 4.7 to 14 wt % of the total dry weight of the composite material is derived from the crosslinking agent.

As will be appreciated, the composite material may be formed as a membrane (e.g. so as to form a skin test platform). In embodiments where the composite material is presented in the form of a membrane it may be functionally identical to the skin test platform described above. As such, all embodiments above relating to the skin test platform material may also be applied here (when the composite material is in the form of a membrane) and so will not be repeated here for brevity, but will be understood to form part of the invention.

The composite material may also be formed from the same materials described herein for the skin test platform material. As such, all materials, amounts, and properties of materials described hereinbefore in embodiments of the skin test platform material (e.g. the crosslinking agent, the silk fibroin's source, the amounts of silk fibroin and crosslinking agent) are as described hereinbefore. In addition, the physical properties of the composite material may be the same as, or similar to, the physical properties described hereinbefore for the skin test platform material (e.g. when provided in the form of a membrane). Given this, these embodiments will not be described here for the sake of brevity but will be understood to form part of the invention.

As will be appreciated, the composite material and the skin test platform material may be formed using similar (or even identical) processing steps. Thus, further aspects of the invention relate to:

(Zi) a method of forming a composite material as described herein, comprising the steps of:

(ia) providing a solution comprising an extracted silk fibroin and a solvent; and

(ib) adding from 5 to 15 wt %, based upon the dry weight of the extracted silk fibroin, of a crosslinking agent to the silk fibroin solution to form the desired composite material by reaction with the silk fibroin; and

(Zii) a method of forming a skin test platform material in the form of a membrane as described herein, comprising the steps of:

(A) providing a solution comprising an extracted silk fibroin and a solvent;

(B) adding from 5 to 15 wt %, based upon the dry weight of the extracted silk fibroin, of a crosslinking agent to the silk fibroin solution to form the skin test platform material in solution by reaction with the silk fibroin;

(C) casting the skin test platform material in solution into a mould and evaporating the solvent to provide the skin test platform material in the form of a membrane.

For the avoidance of doubt, when the composite material is formed in the form of a membrane, it may be formed using the methodology described above for forming the skin test platform. The materials used to form the skin test platform and the composite material are discussed at length hereinbefore and so will not be discussed again here for the sake of brevity.

In embodiments of the invention where the skin test platform, or the composite material in the form of a membrane, mimics human skin structures, the mould used in step (C) above may be one which has a surface that has been shaped to mirror human skin structures, so as to produce a membrane that has a surface that mimics human skin structures.

In embodiments of the methods disclosed above, the reaction of the crosslinking agent with the silk fibroin may be effected using ultrasonication and/or heating for a period of time. When used herein “heating” will be understood to refer to the application of heat to the reaction mixture so as to generate an elevated temperature in the reaction mixture, relative to the ambient conditions. For example, the heating may generate a temperature of from 40 to 80° C., such as from 50 to 70° C., such as about 60° C. in the reaction mixture. A suitable period of time for the heating and/or ultrasonication may be any period of time that allows the reaction to progress to completion and/or to generate the desired properties in the material that is formed. For example, the period of time may be from 10 minutes to 24 hours, such as from 20 minutes to 12 hours, such as from 25 minutes to 1 hour, such as about 30 minutes.

As will be appreciated, if an additive material is to form part of the final product, it may be added to the composite material in step (ib) and/or the skin test platform material in solution in step (B) of the above methods. Suitable additives that may be mentioned herein include, but are not limited to poly(ethylene glycol) diacrylate (PEGDA). For example, after the composite material is formed in step (ib) above (or the skin test platform material in solution in step (B)), the additive may be added as an additive material in any suitable amount to the solution obtained in said steps (e.g. from 1 to 10 wt % of the composite material without the additive material). After the solution containing the additive may be subjected to sonication for a period of time (e.g. from 2 to 60 minutes, such as from 5 to 15 minutes, such as about 10 minutes) before being used to form a film.

Further aspects and embodiments of the invention that may be mentioned herein include those listed below.

In a further aspect, there is disclosed a modified silk fibroin membrane for a skin test platform, comprising:

a) a silk fibroin membrane modified with derivatives of poly(ethylene glycol) (PEG) (wherein an example of the derivatives of PEG is poly(ethylene glycol) diglycidyl ether (PEGDE));

b) wherein the silk fibroin may be obtained from Bombyx marl silkworm silk;

c) the modified silk fibroin membrane may mimic skin absorption behaviour;

e) preferably the modified silk fibroin membrane may have human skin structures.

In yet a further aspect, there is disclosed a method of forming a modified silk fibroin membrane for skin test platform, comprising:

f) providing a solution of extracted silk fibroin;

g) reacting 10 wt % (based on weight of extracted silk fibroin) of derivatives of poly(ethylene glycol) (PEG) with the solution of extracted silk fibroin for a period of time to form a solution of modified silk fibroin;

h) casting the solution of modified silk fibroin in a mould;

i) evaporating solvent of the solution of modified silk fibroin to form a modified silk fibroin membrane;

j) wherein the reacting may comprise either stirring at elevated temperature (of up to 60° C.) or sonicating for 30-45 minutes;

k) preferably wherein the mould is a skin mould allowing formation of skin structures;

l) wherein the derivatives of PEG have functional groups which can react with amino groups;

m) preferably the derivatives of PEG have a molecular weight of less than 1000 Da, preferably about 500 Da; and

n) preferably the derivatives of PEG may be poly ethylene glycol) diglycidyl ether (PEGDE).

As will be appreciated, the skin test platform disclosed herein may be used to replace human or animal skin in tests for suitability of formulations or as a more effective model for the same. Thus, the skin test platforms disclosed herein may be suitable for use in a wide variety of tests as a model system for testing formulations that are to be applied to the skin. Thus, in a further aspect of the invention, there is disclosed a method of determining a property of a test composition, the method comprising the steps of:

(zi) supplying a skin test platform as described hereinbefore;

(zii) applying a test composition to the skin test platform and determining a property of said composition.

Any suitable formulation and property may be determined. Examples of these methods and properties include, but are not limited to, the following.

The method may help determine whether a particular cleansing composition (e.g. a hand wash, a hand sanitizer or a shower gel) has adequate anti-bacterial performance. As such, the method may involve applying an anti-bacterial cleansing composition to the skin test platform, subsequently rinsing it off and then applying a bacterial culture to the skin test platform and measuring the growth of the bacterial culture (or lack thereof) on the skin test platform to determine an antibacterial activity of the anti-bacterial cleansing composition.

The method may relate to determining the absorbency and sensory feel of a product after its application to skin. Thus, the composition may be a skincare product that is applied to a portion of the skin test platform, where the dynamic friction force of the area where the skincare product is applied is then measured.

The method may alternatively relate to measuring the release profile of a perfume. As such, the perfume may be applied to a portion of the skin test platform and the release profile of the perfume over time may then be measured.

Further aspects and embodiments of the invention will; now be described by reference to the following non-limiting examples.

### EXAMPLES

Materials

VITRO-CORNEUM® was purchased from IMS, USA. Poly(ethylene glycol) diglycidyl ether (PEGDE), poly(ethylene glycol) diacrylate (PEGDA), lithium bromide (ACS reagent, ≥99%), lithium chloride (ACS reagent, ≥99%), ninhydrin (ACS reagent), guanidine hydrochloride solution (8 M, pH 8.5, buffered aqueous solution), ethanol (95%) and sodium carbonate (powder, ≥99.5%, ACS reagent) were purchased from Sigma-Aldrich.

Analytical Techniques

All the Fourier-transform infrared (FTIR) spectra were obtained using the FTIR spectrometer (The PerkinElmer Frontier™ IR) with Attenuated Total Reflectance (ATR) accessories from the wavenumber of 4000 cm⁻¹ to 800 cm⁻¹. Before FTIR tests, all the samples were cut into 1 cm² squares and treated in a vacuum oven at 60° C. overnight to remove water inside the protein.

LabRAM Soleil™ Raman spectroscopy was used to measure the penetration of compounds into the silk film.

All nuclear magnetic resonance (NMR) spectra were obtained by dissolving the silk samples in a 1 M LiCl/dimethyl sulfoxide-d₆ solution at 60° C. After 30 min of heating, the sample completely dissolved, and ¹H NMR analysis was performed using BrukerAVIII 400 MHz NMR.

Contact angle measurements were performed using KRUSS Contact Angle (Mobile Surface Analyzer) by dropping a water drop of ˜5 μL on the sample, capturing the real time image when the water drop is deposited on the sample surface, and measuring the water contact angle using the analytical software in the equipment.

The ninhydrin assay protocol is described as follows. Each film (30 mg) of different PEGDE weight ratio (0%/5%/10%/20%/30%) was incubated in 3 mL of ninhydrin solution at 0.35% w/v in ethanol for 1 h at 90° C. under stirring. A blank control was prepared by incubating 3 mL of the same ninhydrin solution under the same conditions.

The amount of free amino groups in the samples can be reflected by the absorbance at 570 nm using UV-Vis-NIR Lambda 950. NH_{without} and NH_{with} are the amounts of free amino groups in pristine SF and in PEGylated SF, respectively. The crosslinking ratio of free amino groups can be calculated with the following equation. A glycine standard solution was used to set up a calibration curve with known free amino group concentrations.

$$crosslinking degree ( \% ) = \frac{{NH}_{without} - {NH}_{with}}{{NH}_{without}} * 100 \%$$

The guanidine hydrochloride assay is described as follows. Each film (10 mg) of different PEGDE ratio (0%/5%/10%/20%/30%) was incubated in 5 mL of 8 M guanidine hydrochloride solution (pH=8) at 60° C. with mild stirring overnight. 1 mL of the solution was taken and evenly mixed with another 2 mL of 8 M guanidine hydrochloride solution (pH=8) to reach a protein concentration of 0.67 mg/mL. The resulting solution was taken for UV-vis experiments. The amount of chromophore tyrosine can be reflected by the absorbance at 270 nm by UV-Vis-NIR Lambda 950. Tyr_{without} and Tyr_{with} are the amounts of tyrosine residues in pristine SF and in PEGylated SF, respectively. The crosslinking degree of tyrosine residues can be calculated with the following equation.

$$crosslinking degree ( \% ) = \frac{{Tyr}_{without} - {Tyr}_{with}}{{Tyr}_{without}} * 100 \%$$

Preparation Method for the Skin Replicate Mould

The skin replicate mould was prepared by coating polydimethylsiloxane (PDMS) on the surface of a rubber skin mould. The PDMS precursor and crosslinker used were from a SYLGARD™ 184 Silicone Elastomer kit purchased from Dow. The PDMS was first prepared by mixing the PDMS precursor and crosslinker at a 10:1 ratio, followed by a thorough mixing process. The prepared mixture of PDMS precursor and crosslinker were poured onto the rubber skin mould and then allowed to cure in 60° C. for 2 h. After PDMS was fully cured, it was peeled off from the rubber skin mould to obtain the skin replicate mould.

### Example 1

The fabrication of the composite material in this example is fully water-based. Raw silk fibers were extracted from Bombyx mori silk cocoons by boiling 20 g silk fibers (also referred to as silk cocoons) in 2 L of 5% Na₂CO₃ aqueous solution. After repetitively washing away residual sericin in the DI water, the degummed SF fiber was obtained. To prepare a SF aqueous solution, the degummed silk fibers were then dissolved in 9.3 M LiBr solution and a clear deep yellow solution was obtained. To remove excess lithium ions and bromide ions, the solution underwent a dialyzing process in DI water and the water was changed in 1 h, 4 h, 8 h, 16 h, 24 h. Protein aggregate were precipitated and a centrifugation process (10,000 rpm, 30 min) was followed to remove protein aggregate, the concentration of the protein solution is determined by weighing the dry weight of solution and is around 5% (w/v). 5 wt %, 10 wt %, 20 wt %, 25 wt % or 30 wt % of PEGDE (average M_{w}: 500 Da) was directly added into silk solution, followed by dynamic stirring and heating at 60° C. for 30 min to obtain PEGDE/SF (used herein to refer to heat-produced composite material) or via an ultrasonication process for 30 min to obtain PEGDE-SF (used herein to refer to sonication-produced composite material). After treatment by heat or sonication, the reacted solution was poured onto the skin replicate mould and put through an ambient evaporation process to obtain a SF/PEGDE substrate with stratum corneum structure. To obtain a SF/PEGDE substrate without stratum corneum structure, the reacted solution can be poured into any flat container (such as petri dish) and the thickness can be adjusted according to the amount of silk solution being added.

Table 1 below shows a list of possible PEG derivatives that allow crosslinking with SF.

TABLE 1
List of possible PEG derivatives that allow crosslinking.
Polyethylene glycol (PEG) derivatives
Poly(ethylene glycol) dithiol
4-arm PEG-Thiol
4-arm PEG-Epoxide
4-arm PEG isocyanate
Isocyanate PEG isocyanate

A comparative membrane, PEGDE+SF, was made as described above except that SF and PEGDE were simply mixed at ambient temperature for 30 min. Another comparative membrane, SF-PEG400, was prepared by reacting PEG (average M_{w}: 400 Da) and SF via the ultrasonication process described above.

Another membrane, PEGDA-SF, was prepared by crosslinking 5 wt % PEGDE (average M_{w}: 500 Da) with SF via the ultrasonication process as described above. Then, 5 wt. % PEGDA (500 g/mol, 0.10 g) was added to the PEGDE-SF solution and the reaction mixture was sonicated for 10 min before pouring it into the mould or any flat container for film formation.

### List of Abbreviations for the Composite Materials Prepared Using Silk Fibroin (SF) and Poly(Ethylene Glycol) Diglycidyl Ether (PEGDE)

PEGDE/SF—heat-produced composite material;

PEGDE-SF—sonication-produced composite material;

PEGDE+SF—simply-mixed PEGDE and SF; and

Silk artificial epidermis—PEGDE-SF without stratum corneum structure

For the avoidance of doubt, use of the above terms in the examples below is consistent with the abbreviations provided above.

Characterization

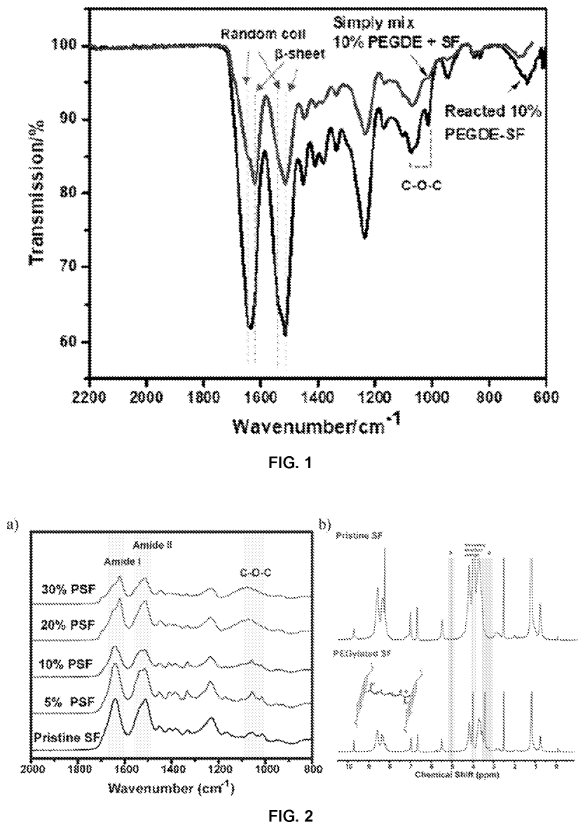

FTIR was used to obtain the characteristic chemical structure of the membrane. The characteristic peak of pristine silk will be presented as amorphous (−1545 cm⁻¹) with some helix structure (−1657 cm⁻¹), silk II crystalline structure (−1627 cm⁻¹ and −1522 cm⁻¹), and their relative change can be shown by relative peak intensity change in the FTIR spectra. While the distribution of the random coil contributes to the stretchability of SF, the crystalline domain determines the strength. As shown in FIG. 1, the random coil domain (1639 cm⁻¹, 1549 cm⁻¹) was found to increase in ultrasonicated PEGDE-SF as compared to simply-mixed PEGDE+SF. Compared to pristine SF, the presence of a 1055 cm⁻¹ peak in the FTIR spectra of PEGDE-SF indicated the inclusion of ether moiety from PEGDE in silk (FIG. 2a). Between pristine SF and 5% PEGDE-SF, there was an obvious shift from β-sheets (1511 cm⁻¹) to random coils (1535 cm⁻¹). This shift persisted to 10% PEGDE-SF, but a continuous increase of PEGDE to 20% and even 30% resulted in a peak shift back to β-sheet. These results confirmed the addition of PEGDE and showed that PEGylation of silk affects its secondary structures which could increase the stretchability as well as the softness of silk.

To characterize the molecular change after cross-linking between PEGDE and silk, 5 wt % to 30 wt % of PEGDE (M_{w}˜500 g/mol) was added to silk and heated at 60° C. for 30 min as described above. ¹H NMR validated that ethylene glycol group was engrafted to SF with the presence of 3.4 ppm (—CH₂—), 5.1 ppm (—CH—) on PEGDE-SF (FIG. 2b).

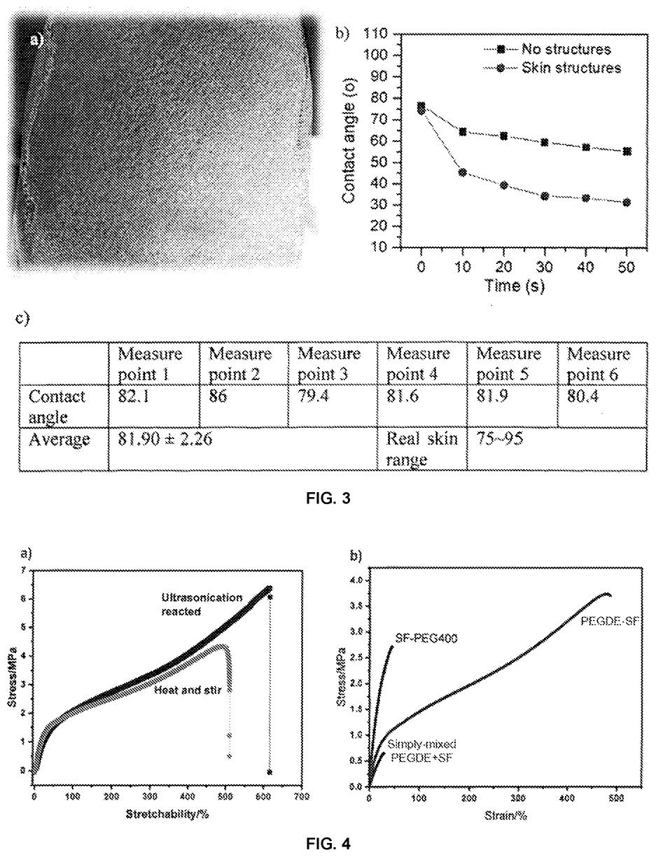

Besides FTIR and ¹H NMR, contact angle measurement of PEGDE/SF was also carried out for surface characterization. According to contact angle tests, PEGDE/SF exhibited a slow absorption behaviour to water with increasing hydrophilicity over absorption (FIG. 3).

The degree of crosslinking between PEGDE and tyrosine residues or free amino group in silk was determined by protein assays and the results are shown in Table 2 and 3.

TABLE 2
Degree of crosslinking between PEGDE and —NH₂ in silk.

| PEGDE wt % | —NH₂ crosslinking degree (%) |
|---|---|
| 5 | 33.9 |
| 10 | 76.3 |
| 20 | 97.7 |
| 30 | 97.7 |

TABLE 3
Degree of crosslinking between PEGDE and -Tyr in silk.

| PEGDE wt % | -Tyr crosslinking degree (%) |
|---|---|
| 5 | 15.0 |
| 10 | 47.5 |
| 20 | 82.6 |
| 30 | 87.4 |

### Example 2

To determine the mechanical strength of the membranes prepared in Example 1, mechanical performance tests were carried out as described below.

The stress-strain curves of PEGDE-SF were obtained by mechanical tester (C42, MTS Systems Corporation) with environmental chamber (Bionix Environbath, MTS Systems Corporation). The circulating water system was connected with environmental chamber for water bath at 37° C. Tensile tests were conducted for more than 3 times at the tensile speed of 2 mm/min. The Young's modulus was calculated using the first 0.5% strain.

Results and Discussion

According to the mechanical test results shown in FIG. 4a, for the same ratio of PEGDE (10% wt), SF reacted with PEGDE via either ultrasonication or stirring at elevated temperature (heat and stir), generated similar mechanical performance. On the other hand, both SF-PEG-400 that does not have active epoxy group and PEGDE+SF prepared by simple mixing exhibited limited stretchability. This can be explained by a lack of crosslinking within the molecular chain of SF in these 2 comparative membranes.

Comparing Young's modulus, PEGDE+SF produced a Young's modulus smaller than PEGDE-SF (FIG. 4b), which can be explained by the strengthening effect of crosslinking between PEGDE and SF. Therefore, only when PEGDE, after reacting via ultrasonication treatment or stirring at elevated temperature with SF, can the crosslinking reaction happen. Nonetheless, SF-PEG-400 showed an even better strength enhancement (FIG. 4b) which might result from its excess hydroxyl group, according to a previous publication that mentioned that the presence of hydroxyl group can add to the crystallinity of SF (X. Wang et al., Acta Biomater. 2015, 12, 51-61).

### Example 3

The surface hydrophobicity of PEGDE/SF with stratum corneum structure (prepared in Example 1) was tuned by varying the PEG derivative ratio, reacted silk solution concentration, and the type of PEG derivative used. The reaction was induced by the chemical input amount as presented in the Table 4. The surface hydrophobicity of the prepared samples was determined by water contact angle measurements.

TABLE 4
Chemical input of three methods that modify the hydrophobicity of
silk artificial epidermis.

| | | |
|---|---|---|
| Amount of silk | Silk concentration | Amount of PEGDE |
| (g) | (wt. %) | (g) |

Varying the PEG derivative ratio

| | | |
|---|---|---|
| 1 | 5% | 0.05 |
| 1 | 5% | 0.10 |
| 1 | 5% | 0.20 |
| 1 | 5% | 0.30 |

Varying the reacted silk solution concentration

| | | |
|---|---|---|
| 1 | 3% | 0.10 |
| 1 | 6% | 0.10 |
| 1 | 10% | 0.10 |

Results and Discussion

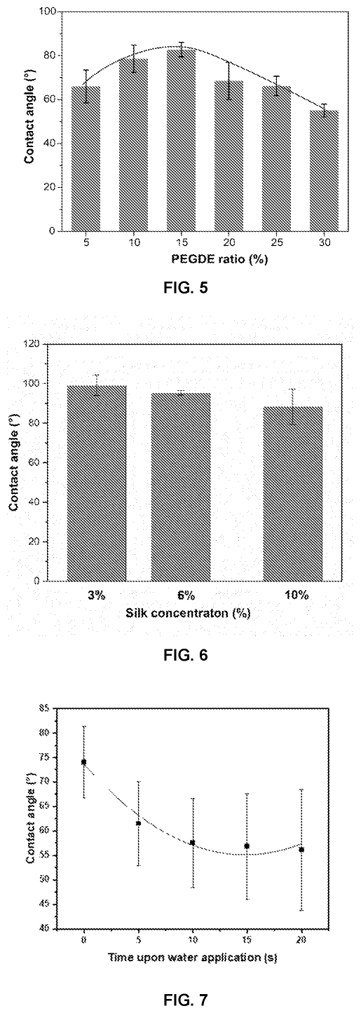

The impact of PEGDE weight ratio to hydrophobicity is shown in FIG. 5. The water contact angles for different PEGDE ratios showed that the PEGDE-SF film first became more hydrophobic with 5% to 10% PEGDE addition, then turned more hydrophilic beyond 20% PEGDE addition. In general, from 5% to 30%, a contact angle range of 70°-85° was obtained.

The water contact angles of PEGDE/SF with stratum corneum structure fabricated from different silk concentrations (3%, 6% and 10%) also showed that as the concentration of silk solution increases, the contact angle gradually decreases (FIG. 6). This could be attributed to the different aggregate of protein chain during the film forming process. A contact angle range of 75°-105° was achieved by tuning the concentration of the reacted silk solution.

The surface wettability of PEGDA-SF was also investigated. It was found that the PEGDA-SF with stratum corneum structure exhibited a more hydrophilic property with a lower contact angle of 74.07±7.26° (FIG. 7).

### Example 4

To determine the effect of the ratio of PEGDE to SF on the stretchability of the PEGDE-SF membranes prepared in Example 1, mechanical tests as described in Example 2 were carried out, and the results are provided below.

Results and Discussion

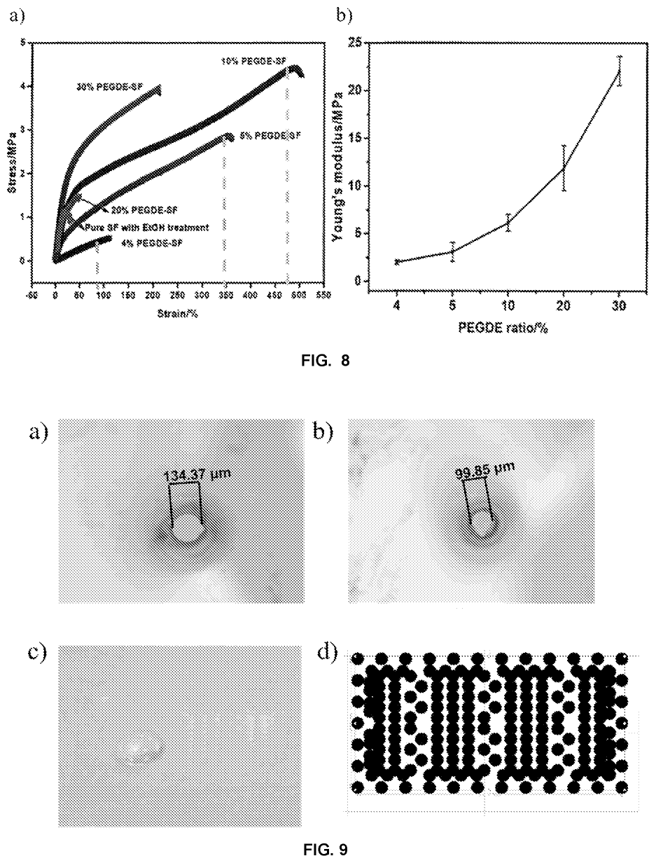

FIG. 8a shows the results of the mechanical tests of PEGDE-SF with different weight ratio of PEGDE to SF. As the amount of PEGDE increased, the Young's modulus of the resulting PEGDE-SF membranes increased (FIG. 8b). The resulting PEGDE-SF membranes with 10% PEGDE addition had Young's modulus values that are similar to human skin. Accordingly, all the PEGDE-SF membranes prepared in Example 1 resembled human skin as theoretically, human skin can be stretched up to 100% strain. 10 wt % PEGDE-SF was selected for further studies because at this ratio, the stretchability almost reached 500%. Therefore, all of the following examples use a 10% wt of PEGDE to SF or 10% wt of PEG400 to SF.

### Example 5

Besides tuning of the surface hydrophobicity, skin pores are also important in evaluating the interaction of skin with personal care products. In general, skin pores can range from 100 to 500 μm with a density of 50-80 pores cm⁻². Micropores that mimic the density and size of skin pores can be generated and printed on skin test platforms using standard electronic cutting machine as shown in FIG. 9a-d. Therefore, PEGDE/SF with and without stratum corneum structure (prepared in Example 1) were taken for water contact angle measurements.

Results and Discussion

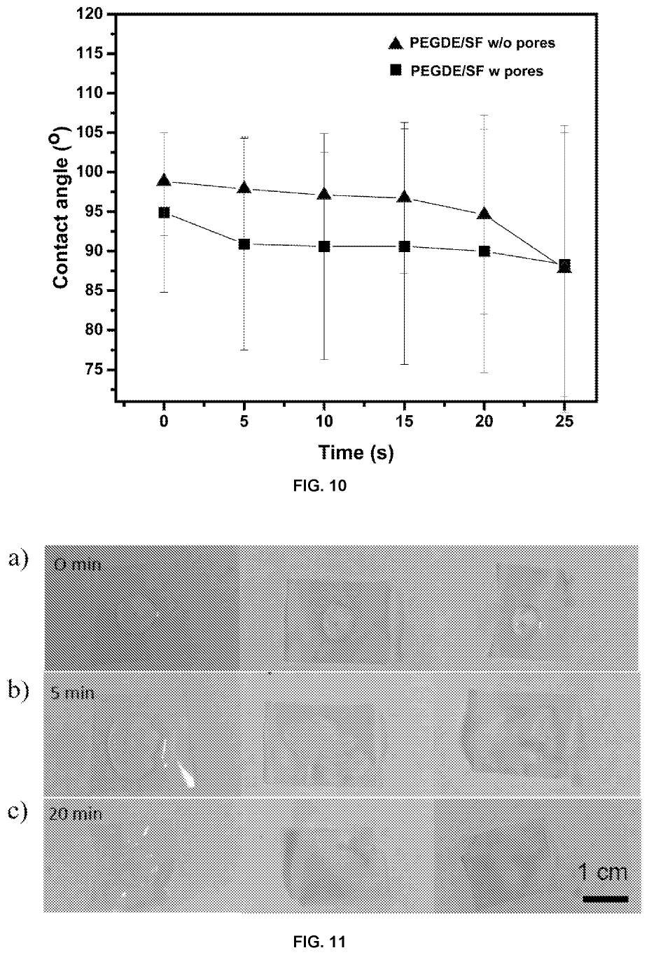

It was found that the contact angles for PEGDE/SF with pores were higher than PEGDE/SF without pores (FIG. 10). However, the PEGDE/SF with pores was more hydrophilic and absorbed water faster than PEGDE/SF without pores.

### Example 6

The silk artificial epidermis prepared in Example 1 were taken for toner and cream absorption studies. As niacinamide and glycerin are two skincare ingredients present in most skincare products, the penetration of niacinamide and glycerin into silk artificial epidermis was evaluated. The experimental protocol and results are provided below.

The silk artificial epidermis was first immersed in water for 10 min. Then, the silk artificial epidermis was taken out and wiped with a wiper to remove surface water. Next, niacinamide or glycerin was applied on the surface of the silk artificial epidermis and left to sit for 30 min or 4 h. Finally, the resultant sample was taken for cross-sectional Raman spectroscopy to determine the penetration of the chemicals into silk artificial epidermis.

Results and Discussion

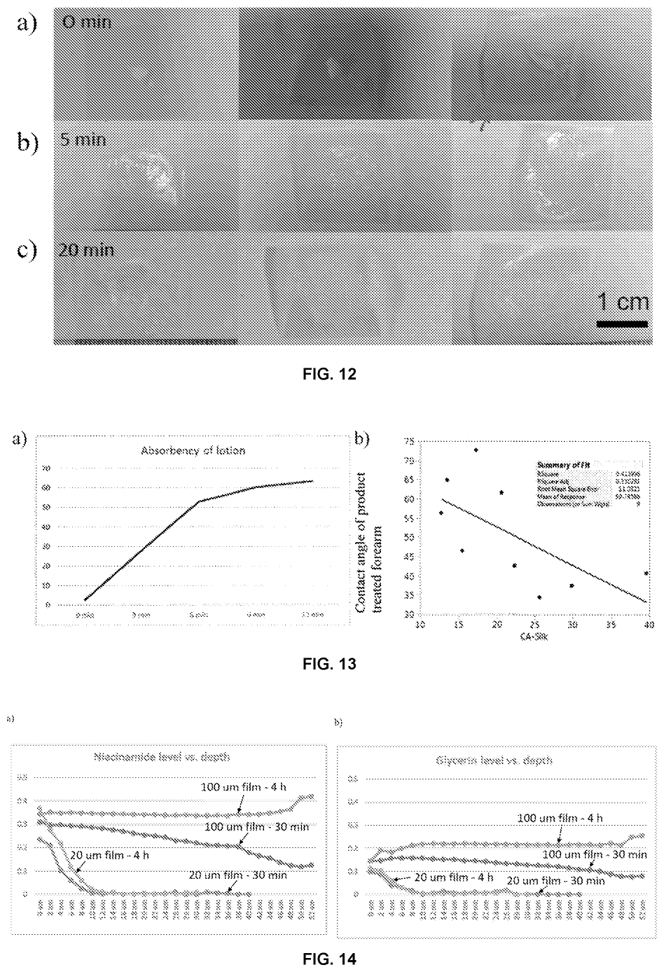

The absorption behaviour of silk artificial epidermis towards lotion is very similar to human stratum corium (FIG. 11a-d). The first to be absorbed would be small molecules like water with a very fast absorption rate. Next, relatively larger oil-based molecules would be absorbed at a slower rate than water. Lastly, the remaining content in the toner would be fully absorbed. Besides toner, the absorption behaviour of silk artificial epidermis towards cream was also studied. The silk artificial epidermis showed a slow absorption of both toner and cream over time (FIG. 12a-d). After complete absorption of both the toner and cream, no residue was observed on the surface of silk artificial epidermis.

The absorbency of lotion by silk artificial epidermis is shown in FIG. 13a. A correlation was built between lotion or cream residue on forearm and contact angle of silk artificial epidermis (FIG. 13b). This relationship can provide further quantitative relationship to study the absorption behaviour of silk artificial epidermis.

The penetration depth of skincare products is closely related to their moisture retention performance. Both niacinamide and glycerin were found to penetrate into half-depth of the silk artificial epidermis for different time duration (30 min and 4 h), indicating a continuous absorption behaviour similar to real skin (FIG. 14).

### Example 7

To determine the anti-bacterial property of silk artificial epidermis prepared in Example 1, bacteria culture tests were carried out as described below.

Staphylococcus aureus (ATCC 13368) and Escherichia coli 0157:H7 (ATCC 700728) were revived from −80° C. storage, followed by incubation at 37° C. for 18 h. 5 ml of the bacteria suspension was seeded onto the surface of the tested film. After 24 h incubation, the surface was observed by SEM, and Image J software was employed for the quantification of the bacteria.

Results and Discussion

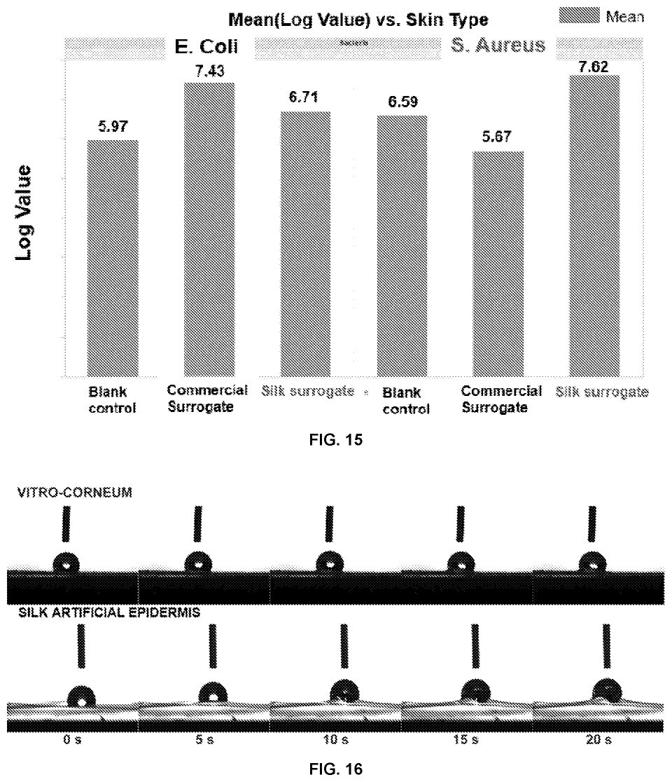

Bacteria culture tests on silk artificial epidermis showed that silk artificial epidermis was susceptible to bacteria growth (FIG. 15). This is alike to human skin where bacteria growth will cause some illness to human body. Therefore, a potential application here is for companies to test anti-bacterial skin products, such as body shampoo, hand wash, etc., on this skin test platform.

### Example 8

To further understand the interaction of PEGDE and silk at the molecular level, simulations on a representative model of a single PSF molecular chain were performed as described below.

A 44-residue silk peptide model was built based on a previous report (Zhou, C.-Z. et al., Proteins 2001, 44, 119-122), in which PEGylation was designated to 1 tyrosine (TYR) and 1 asparagine (ASN) residue because both amino acids side chain holds the highest mol % of phenol and free amine group, respectively, in silk (Murphy, A. R. et al., J. Mater. Chem. 2009, 19, 6443-6450). The molecular dynamics simulation was carried out in an aqueous environment.

Among the residue sequences, residues in N-terminal, C-terminal and linkers are categorized as amorphous, as opposed to crystalline domain. The molecular model of silk was built on the initial structures of the amorphous domain based on the single SF sequence according to Linker 6 published by Zhou et al. (Zhou, C.-Z. et al., Proteins 2001, 44, 119-122): (GAGAGAGAGAGTGSSGFGPYVANGGYSGYEYAWSSESDFGTGS). The peptide chain was constructed using Software package SYBYL 8.0 (Tripos Associates, Inc.), and the PEGDE model was constructed and optimized with Gaussian 09 using 6-31G* basis set. TYR holds the highest Mol % in residues with phenol group. ASN also accounts for higher Mol % in residues with free amine. Therefore, to create a simple model to illustrate the influence of PEGylation to SF molecular chain, PEGylation were designated to 1 TYR and 1 ASN residue in the 44-residue peptide model, respectively. After optimization, the PEGDE side chain was grafted onto silk peptide chain in ASN23 and TYR29, respectively. This process was done in GaussView and the residue topology parameters (RTP) for residue 23 and 29 were changed according to the structure after PEGylation. After the construction of the PEGylated peptide model, energy minimization was performed on the model to optimize the structure. The following molecular dynamics simulation was carried out in water, where the systems were first solvated in a water box of the dimension 95.43×127.8×70.33 nm³. The extended simple point charge model (SPC/E) was adopted for water. Then, three Na ions were added to replace some of the water molecules to neutralize the system. Finally, molecular dynamics simulation was performed to the pre-equilibrated systems under 100 ns of Canonical ensemble (NVT) at a temperature of 300 K and 100 ns Isothermal-isobaric (NPT) ensemble at a temperature of 300 K and pressure of 1 bar in water.

Secondary structures of the protein were analyzed using the STRIDE algorithm, which is a built-in module in the VMD Molecular Graphics Viewer. All the simulations were conducted by the software package GROMACS using Amber 03 force field. Solvent accessible surface area (SASA), per-residue-area and energy were also analyzed using Gromacs. All the representative snapshots for protein configuration were produced using VMD Molecular Graphics Viewer.

Results and Discussion

Simulation results showed that the PEGylated silk chain was dominated by extensible secondary structures (random coils and turns). The quantitative secondary structure distribution in the water at equilibrium showed that the percentage of the random coil in pristine SF and PSF were 43.2% and 54.6%, respectively, while the percentage of turns were both approximately 45.4% (Table 5). This transition in the secondary structure between PSF and pristine SF is consistent with the increase in the stretchability of PSF as shown in the FTIR spectra and mechanical stretching test presented in Examples 2 and 3.

TABLE 5
Quantitative distribution of secondary structures in water for pristine
SF and PSF

| | Secondary structure (%) | | | |
|---|---|---|---|---|
| Sample | Coil | Turn | Helix | Beta-sheet |
| Pristine SF | 43.18 | 45.46 | 6.82 | 4.54 |
| PSF | 54.55 | 45.45 | 0 | 0 |

### Comparative Example 1

The water absorption ability of silk artificial epidermis (prepared in Example 1) and commercial product VITRO-CORNEUM® was compared. The thickness of both films in the experiment was 30 μm.

Results and Discussion

Within 20 s of exposure to water, the silk artificial epidermis showed an immediate absorption from 5 s while for VITRO-CORNEUM®, there was very limited absorption (FIG. 16). The contact angle was not used as a quantitative data here because upon absorbing, the film surface formed wrinkles that brought deviation to the standard measurement of contact angle.

### Example 9

Following the promising results in previous examples, a larger scale silk artificial epidermis was prepared for patch study. The patch was prepared as described in Example 1, and the thickness distribution and size of the film are as follows: edge: 74.8±14.08 μm; centre: 74.3±13.20 μm; and size: 35 cm*20 cm.

Results and Discussion

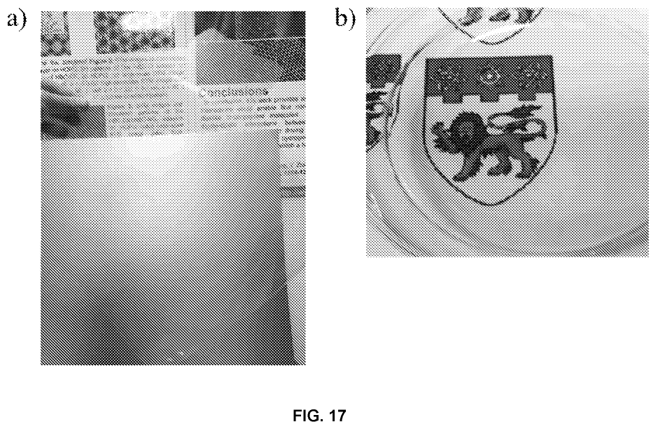

The obtained large-area silk artificial epidermis film (FIG. 17a) showed high transparency (FIG. 17b).

1. A skin test platform material in the form of a membrane, said material comprising:
silk fibroin; and
a crosslinking agent that forms crosslinks in the silk fibroin,
wherein from 4.7 to 14 wt % of the total dry weight of the skin test platform material is derived from the crosslinking agent.

2. The skin test platform material according to claim 1, wherein the membrane has a surface that has been shaped to mimic human skin structures.

3. The skin test platform material according to claim 1, wherein the membrane has a plurality of pores.

4. The skin test platform material according to claim 1, wherein the crosslinking agent is selected from one or more of the group consisting of O′O-bis[2-(N-succinimidyl succinylamino) ethyl]polyethylene glycol (NHSP), glutaraldehyde, poly(ethylene glycol) dithiol, a 4-arm PEG-thiol, a 4-arm PEG-epoxide, a 4-arm PEG isocyanate, isocyanate PEG isocyanate, and poly(ethylene glycol) diglycidyl ether.

5. The skin test platform material according to claim 4, wherein the crosslinking agent is poly(ethylene glycol) diglycidyl ether.

6. The skin test platform material according to claim 4, wherein when the crosslinking agent is a polymeric material it has a number average molecular weight of from 150 to 2,000 Daltons.

7. The skin test platform material according to claim 1, wherein one or both of the following apply:
(az) the membrane is from 50 to 500 μm thick; and
(bz) from 6.5 to 11 wt % of the total dry weight of the skin test platform material is derived from the crosslinking agent.

8. The skin test platform material according to claim 1, wherein the skin test platform further comprises an additive material.

9. The skin test platform material according to claim 1, wherein one or more of the following apply:
(a) the silk fibroin is derived from Bombyx mori silkworm silk;
(b) the skin test platform material can be subjected to an elongation strain of from 300 to 550% without breaking;
(c) the skin test platform material has a Young's modulus of from 2.5 to 10 MPa;
(d) when the skin test platform material has not been shaped to mimic human skin structures, it has a water contact angle at 30 seconds after contact with water of from 60 to 70°;
(e) when the skin test platform material has been shaped to mimic human skin structures, it has a water contact angle at 30 seconds after contact with water of from 30 to 40°;
(f) a difference between a water contact angle of the skin test platform material that has not been shaped to mimic human skin structures and a water contact angle of the same skin test platform material that has been shaped to mimic human skin structures at 30 seconds after contact with water is from 20° to 40°; and
(g) when the skin test platform material has not been shaped to mimic human skin structures, it has a water contact angle upon immediate contact with water of from 80 to 90°.

10. A composite material comprising:
silk fibroin; and
a crosslinking agent that forms crosslinks in the silk fibroin,
wherein from 4.7 to 14 wt % of the total dry weight of the composite material is derived from the crosslinking agent.

11. The composite material according to claim 10, wherein the composite material is formed as a membrane.

12. The composite material according to claim 10, wherein the crosslinking agent is selected from one or more of the group consisting O′O-bis[2-(N-succinimidyl succinylamino) ethyl]polyethylene glycol (NHSP), glutaraldehyde, poly(ethylene glycol) dithiol, a 4-arm PEG-thiol, a 4-arm PEG-epoxide, a 4-arm PEG isocyanate, isocyanate PEG isocyanate, and poly(ethylene glycol) diglycidyl ether.

13. The composite material according to claim 12, wherein the crosslinking agent is poly(ethylene glycol) diglycidyl ether.

14. The composite material according to claim 12, wherein one or both of the following apply:
(aaz) when the crosslinking agent is a polymeric material it has a number average molecular weight of from 150 to 2,000 Daltons; and
(bbz) from 6.5 to 11 wt % of the total dry weight of the composite material is derived from the crosslinking agent.

15. The composite material according to claim 10, wherein the composite material further comprises an additive material.

16. (canceled)

17. A method of forming a composite material according to claim 10, comprising the steps of:
(ia) providing a solution comprising an extracted silk fibroin and a solvent; and
(ib) adding from 5 to 15 wt %, based upon the dry weight of the extracted silk fibroin, of a crosslinking agent to the silk fibroin solution to form the desired composite material by reaction with the silk fibroin.

18. A method of forming a skin test platform material in the form of a membrane according to claim 1, comprising the steps of:
(A) providing a solution comprising an extracted silk fibroin and a solvent;
(B) adding from 5 to 15 wt %, based upon the dry weight of the extracted silk fibroin, of a crosslinking agent to the silk fibroin solution to form the skin test platform material in solution by reaction with the silk fibroin;
(C) casting the skin test platform material in solution into a mould and evaporating the solvent to provide the skin test platform material in the form of a membrane.

19. (canceled)

20. The method according to claim 17, wherein the method further comprises applying a plurality of pores to the membrane.

21. (canceled)

22. (canceled)

23. (canceled)

24. A method of determining a property of a test composition, the method comprising the steps of:
(zi) supplying a skin test platform as described in claim 1;
(zii) applying a test composition to the skin test platform and determining a property of said composition.

25. (canceled)

26. The method according claim 18, further comprises applying a plurality of pores to the membrane.
